(19) United States
(12) Patent Application Publication
(10) Pub. No.: US 2019/0076607 A1
(43) Pub. Date: Mar. 14, 2019

(54) INHALER AND MESH FOR AN INHALER

(71) Applicant: LUPIN ATLANTIS HOLDINGS SA, Zug (CH)

(72) Inventors: Xian-Ming ZENG, Coral Springs, FL (US); Michael GOLLER, Zug (CH); Thommandru Vijaya KUMAR, Pune (IN); Raghavendra NAYAK, Pune (IN); Mahesh BAVISKAR, Pune (IN); Ganesh VEKHANDE, Pune (IN)

(21) Appl. No.: 15/703,574

(22) Filed: Sep. 13, 2017

(51) Int. Cl. A61M 15/00; A61K 9/00; A61K 31/439

(52) U.S. Cl. CPC A61M 15/0085; A61M 15/0021; A61M 15/003; A61M 2202/30; A61K 31/439; A61M 2202/064; A61K 9/0075

(57) ABSTRACT

An inhaler device for dispensing medicament comprising: a dispenser flow channel leading to a mouthpiece, a medicament holder, and a mesh part placed in the dispenser flow channel between the medicament holder and the mouthpiece, wherein said mesh part is made of a Polyoxymethylene.

### FIELD OF THE INVENTION

The present invention relates to a dry powder inhaler for dispensing medicament for inhalation.

### BACKGROUND OF THE INVENTION

Dry powder inhalers (DPIs) are an important and increasingly investigated method of modern therapy for a growing number of respiratory diseases. DPIs are a promising option for certain patient populations, and may help to overcome several limitations that are associated with other types of inhalation delivery systems (e.g., accuracy and reproducibility of the dose delivered, compliance and adherence issues, or environmental aspects). Today, more than 20 different dry powder inhalers are on the market to deliver active pharmaceutical ingredients (APIs) for local and/or systemic therapy.

DPI devices are designed to reproducibly deliver a predefined dose of a drug to the small airways and alveolar region of the lung. It is well reported that particles with a mass median aerodynamic diameter (MMAD) of 1-5 μm are effectively deposited at aforementioned sites. The MMAD of a particle depends on its geometrical diameter, density, and morphology with these properties generally being manipulated during the manufacturing process. Due to inter-particulate forces, i.e., mechanical interlocking, capillary, electrostatic, and van der Waals forces, micronized powders are very adhesive/cohesive, spontaneously forming agglomerates.

DPIs that utilize a patient's inspiratory airflow to provide the required energy to overcome the aforementioned inter-particulate forces are known as “passive” devices, whereas those that utilize other sources of energy are referred to as “active” devices. One advantage of utilizing a patient's inspiratory airflow as the main source of energy is that such devices are breath actuated; this inherently avoids the need to synchronize the actuation and inspiration maneuver by the patient. The downside of this approach is that devices currently available show a device-specific airflow resistance, and this often demands a relatively high inspiratory effort which might be a hurdle for patient populations suffering from obstructive airway diseases such as asthma or COPD, the elderly, or very young. The extent of lung deposition is also dependent on the individual patient's inspiratory flow rate causing a potential difference in the dose effectively delivered due to this variability.

The powder properties such as particle size, morphology, shape, and material, as well as on environmental factors, e.g., relative humidity also play an important role to deliver an effective inhalation dose. Since the extent of agglomeration negatively affects the fraction of the inhaled powder, which is within the respirable range, these agglomerates must be effectively deagglomerated prior to or during the processes of aerosolization and inhalation.

Since flow properties of micronized powders are often poor, most formulations consist of physical blends of drug particles with larger (30-90 μm) carrier particles such as lactose, to aid deagglomeration and powder flow. In light of the aforementioned considerations, the ideal DPI would reproducibly deliver an accurate dose, regardless of a patient's condition.

WO 2011/129787 A1 discloses a powder inhaler for delivery of medicament in dry powder form characterized in that each component of said device is coated with antistatic substance and/or antibacterial substance.

WO 2002/028368 A1 discloses an inhaler device that may comprise one or several parts made from polyoxymethylene (POM).

US 2011/0297151 A1 discloses an inhaler device with various parts and the plastics from which individual parts of the inhaler are produced are polymers, thermoplastic polycondensates, polyadducts, modified natural substances or rubbers or mixtures of these plastics. Preferred are polyolefins, vinyl chloride polymers, styrene polymers, polyacetals, polyamides, thermoplastic polyesters and polyarylethers or mixtures of these plastics. Examples of these plastics are polyethylene, polyvinyl chloride, polyoxymethylene, polyacetal, acrylonitrile/butadiene/styrene; (ABS), acrylonitrile/styrene/acrylic ester (ASA), polyamides, polycarbonate, poly(ethyleneterephthalate), poly(butyleneterephthalate) or poly(phenylene ether).

WO2015/128789 A1 discloses an inhaler device and the device may be made from any suitable material. Preferably the device is made of plastic, for example ABS (acrylonitrile butadiene styrene), PC (polycarbonate), PA (polyacetal) or PS (polystyrene), or mixtures thereof, or of an antistatic material such as delrin or stainless steel.

U.S. Pat. No. 9,010,323 discloses an inhaler having a sieve part for administrating medicament. The inhaler device having a suction air channel leading to a mouthpiece, a substance supply container that is moveable inside a receiving chamber and the sieve part disposed in the suction air channel between the receiving chamber and the mouthpiece, wherein the sieve part includes a retaining edge, a sieve area contained in a cross sectional area within the retaining edge, and a protruding area that protrudes to one side and has a flat portion.

In general, the powdered inhalation devices are used for inhaling either single or multi-dose of powdered medicament from capsules. The devices are configured to have medicament holders which hold the capsules containing the powdered medicament. A piercing mechanism provided with the device pierces the capsule and enables the medicament to get dispersed into the air sucked by the user during the process of inhalation. The inspiratory force makes capsule vibrate or creates a turbulence making possible to empty capsule containing cohesive powders and dispense it through mesh and mouthpiece. The emptied capsule remains in the device which is then discarded prior to the next use of the device.

It is potentially desirable that inhalation device delivers sufficient amount of the medicament to the patient for inhalation. The ability to discharge the contents of the capsule and the residual amount of content in the inhalation device determines the efficiency of the inhalation device. It is also potentially desirable that mesh part, placed in dispenser flow channel held by mesh holder, in inhalation device offers improved fine particle fraction for inhalation and decreased mass median aerodynamic diameter. The mesh part and mesh holder may be molded into a single component for the ease of manufacturer. At the same time, it assists in spinning motion of capsule in medicament holder to provide high quality aerosol containing high amount of fine particles.

Depending on the mechanism of deagglomeration, aerosolization, dose metering accuracy, and the inter-patient variability, dry powder inhalers demonstrate varying performance levels. With the wide variety of applications related to specific APIs, there exists a range of different devices with distinct features.

The present invention discloses an inhaler device with a mesh part made up of Polyoxymethylene(POM) for inhaling the powder medicament from a capsule with an improved dispersion and de agglomeration of dry powder formulation.

### SUMMARY OF THE INVENTION

According to one aspect of the present invention there is provided an inhaler device for dispensing medicament comprising: a dispenser flow channel (10) leading to a mouthpiece (4), a medicament holder (5), and a mesh part (7) placed in the dispenser flow channel (10) between the medicament holder (5) and the mouthpiece (4), wherein said mesh part (7) is made of a Poly oxy methylene.

In an another aspect of the invention the inhaler device for dispensing Tiotropium or pharmaceutically acceptable salts thereof comprises: a dispenser flow channel (10) leading to a mouthpiece (4), a medicament holder (5), and a mesh part (7) placed in the dispenser flow channel (10) between the medicament holder (5) and the mouthpiece (4), wherein said mesh part (7) is made of a Poly oxy methylene.

In a specific embodiment of the invention the inhaler device for dispensing Tiotropium Bromide comprises: a dispenser flow channel (10) leading to a mouthpiece (4), a medicament holder (5), and a mesh part (7) placed in the dispenser flow channel (10) between the medicament holder (5) and the mouthpiece (4), wherein said mesh part (7) is made of a Poly oxy methylene.

### BRIEF DESCRIPTION OF THE DRAWINGS

FIG. 1 shows a cross-section view of an inhaler having a mesh part arranged in the dispenser flow channel.

FIG. 2 shows a cross-section view of the mesh part.

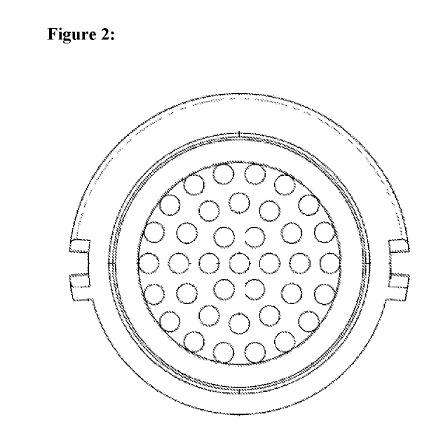

### DETAILED DESCRIPTION

The present invention relates to an inhaler device for dispensing medicament comprising: a dispenser flow channel (10) leading to a mouthpiece (4), a medicament holder (5), and a mesh part (7) placed in the dispenser flow channel (10) between the medicament holder (5) and the mouthpiece (4), wherein said mesh part (7) is made of a Polyoxy methylene.

Generally, a sufficient amount of medicament has to be absorbed in the lungs for an effective inhalation. Delivery and absorption of a sufficient amount of medicament in the lungs of the patient is possible when the dry powder medicament that is inhaled has a uniform and homogeneous particle size distribution. In dry powder inhalation, agglomerates are formed of the dry powder medicament in the capsule due to factors such as moisture rate of the capsule, electrostatic forces, energy exchange between dry powder particles, etc. These agglomerations affect the particle size distribution of dry powder medicament in the capsule. Inhalation of a dry powder medicament which does not have a uniform or homogenous particle size distribution results in failure to deliver a sufficient amount of medicament to patient.

The powdered medicament particles suitable for delivery to the bronchial or alveolar region of the lung have an aerodynamic diameter of less than 10 micrometers, preferably less than 6 micrometers. Coarse particles i.e. particles having larger aerodynamic diameter may get caught in other portions of the respiratory tract, such as the nasal cavity, mouth or throat before reaching the patient's lungs during inhalation. Since the agglomerates formed in the dry powder medicament in the capsule may be in large sizes, they may lead to failure to deliver sufficient amount of medicament to patient. To this respect, these agglomerates formed in the dry powder medicament have to be disaggregated in the inhaler before delivery to the patient.

Surprisingly it has been found that the effective turbulence created in the medicament holder during inhalation that lead to improved drug dispersion and higher fine particle fraction and effective inhalation in the case that the mesh part is made of Polyoxymethylene.

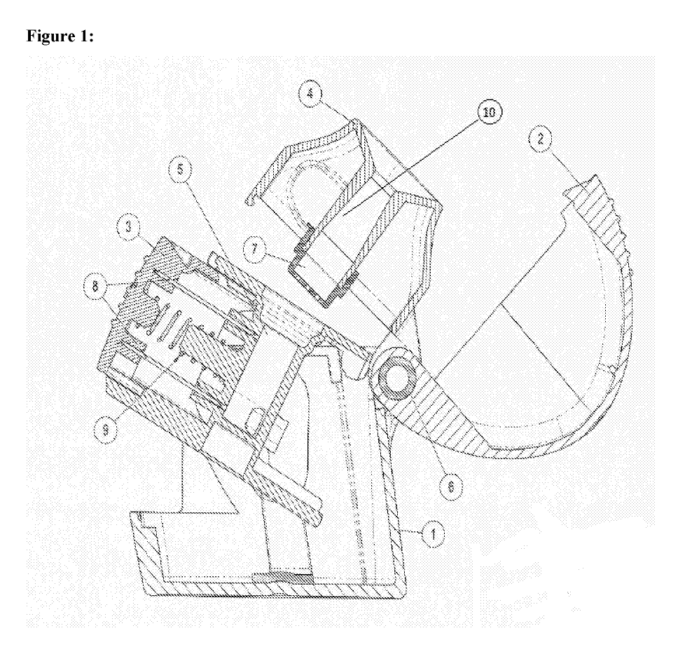

Referring to FIG. 1, a powder inhaler as basically known from the previously mentioned WO 2015/128789 A1 is shown in cross-section. For further details, see the above-mentioned publication the contents of which are hereby incorporated by reference, including information as to the incorporation of features from the above-mentioned publication in a claim of the present application.

In detail, the basic components in medicament dispenser has a housing (1), lid (2), an actuating button (3), a mouthpiece (4), a medicament holder (5), axis (6), mesh part (7), needle (8), spring (9), dispenser flow channel (10) and mesh holder (11).

Further, adjoining the mouthpiece (4) on the inside is a dispenser flow channel (10) which merges into a medicament holder (5) in which there is a medicament carrier. Between the medicament holder (5) and the dispenser flow channel (10) is provided a mesh part (7) held in dispenser flow channel (10) by mesh holder (11).

The present invention provides a mesh part, in the device disclosed in WO 2015/128789, is made up of Polyoxymethylene. Mesh made up of Polyoxymethylene improves dispersion and deagglomeration of dry powder formulation during inhalation. It also contributes to continuous spinning motion of capsule in the medicament holder aligned to airflow direction.

Polyoxymethylene is a low surface energy material and the use of mesh made up of Polyoxy methylene improves fine particle fraction (FPF) and decrease MMAD of the emitted aerosol by reducing the electrostatic attraction.

The inhaler is operated as disclosed in the above mentioned publication to pierce the capsule, during which external air enters the medicament holder through air inlet upon inhalation by patient to create turbulence in air flow entraining dry powder medicament in the inhaler.

Dry powder inhaler, as disclosed herein, have a variety of structural configurations and can be used for dispensing powders or capsules or mixtures thereof for pulmonary or oral administration.

In some embodiments, the dry powder inhaler comprises a medicament holder (5) to house the medicament carrier. In a more specific embodiment, the medicament carrier carrying medicament portion is in the form of a capsule.

In some embodiments, mesh part (7) is held in the dispenser flow channel (10) by mesh holder (11). In further embodiment, the mesh part (10) is placed in between the medicament holder (5) and the mouthpiece (4).

In some embodiments, the dispenser flow channel (10) can be in the form of exit channel if the medicament carrier is in solid form such as capsules.

The term ‘mouthpiece’ is used to mean an element through which a patient may inhale a medicament. In one aspect, the inhalation is by oral means with the patient placing the mouthpiece in the mouth.

The term “effective turbulence” refers to the turbulence which ensures disaggregation of any agglomerates formed in the medicament in dry powder form entrained by the air flow entering the capsule chamber of the inhaler upon inhalation by patient.

The term “effective inhalation” refers to an inhalation in which a sufficient amount of medicament required for an effective treatment is delivered to the patient's lungs.

The outlet of the medicament holder of an inhaler of the present invention ensures the creation of turbulent air flow facilitating the disaggregation of the agglomerates formed in the dry powder medicament and providing effective delivery to the patient. The turbulence created in the air flow entraining dry powder medicament leads to pressure increase at the outlet of the medicament holder, in the area where the capsule chamber is in the center. So, the pressure here gets higher compared with the pressure at the outlet of the mouthpiece and this pressure difference eases delivery of the air flow entraining dry powder medicament since the dry powder medicament moves from high pressure to low pressure. In addition, due to the turbulence created in the air flow entraining dry powder medicament, agglomerates in the dry powder medicament are disaggregated and the dry powder medicament entrained by the air flow is delivered to the patient.

The mesh part made of polyoxymethylene prevents the accumulation of medicament particles at the outlet of the medicament holder and thus at the inlet of the dispenser flow channel caused due to electrostatic forces in addition to provide for the disaggregation of the agglomerates in the dry powder medicament entrained by the air flow entering the medicament holder. After the air flow entraining the dry powder medicament exits the medicament holder, it passes through the dispenser flow channel and the mouthpiece of the inhaler and reaches the patient to deliver dry powder medicament with a uniform and homogenous particle size distribution.

The inhaler of the present invention is composed of various components in order to ensure inhalation of the dry powder medicament contained in the capsule.

In some embodiments, the medicament may be delivered alone or delivered together with one or more excipients or carriers which are suitable for inhalation. Suitable excipients or carriers include but not limited to organic excipients such as polysaccharides (i.e. starch, cellulose and the like), lactose, glucose, mannitol, amino acids, and maltodextrins, and inorganic excipients such as calcium carbonate, magnesium stearate, Sodium stearyl fumarate, and sodium chloride. In preferred embodiment, carrier or excipient is lactose.

Particles of powdered medicament and/or excipient may be produced by conventional techniques, for example by micronisation, milling or sieving. Additionally, medicament and/or excipient powders may be engineered with particular densities, size ranges, or characteristics.

Particles may comprise active agents, surfactants, wall forming materials, or other components considered desirable by those of ordinary skill.

The inhaler device described herein can be used for the treatment and prophylaxis of respiratory diseases, including but not limited to asthma, chronic obstructive pulmonary disease (COPD) bronchitis or chest infection.

The inhaler device can be a hand-held dispenser and can be operated by a single hand. The inhaler device can be of circular form, non-circular form, an elongate form, an elliptical form, or other shapes. The inhaler device can include a flat surface or a resting surface. At least a portion of the inhaler device can be shaped for ease of grip by the user. The inhaler device can include a housing or a diskette assembly.

In some embodiments, the housing can include at least one piercing element for puncturing the capsule. In a more specific embodiment, the housing can include at least two piercing elements.

In some embodiments, the capsule comprises medicament alone or delivered together with one or more excipients or carriers which are suitable for inhalation. In some embodiments, the capsule comprises medicament comprising a single active pharmaceutical ingredient. In some embodiments, the capsule comprises medicament comprising two or more active pharmaceutical ingredients.

In some embodiments, the inhaler is composed of various components made of same or different material including but not limited to acrylonitrile butadiene styrene (ABS), polycarbonate/acrylonitrile butadiene styrene terpolymer blend (PC/ABS), polyoxymethylene, nylon and silicone rubber.

In some embodiments, the term capsule is intended to be understood broadly and includes any suitable receptacle for powdered pharmaceutical compositions. The capsule may be formed from any suitable material, including gelatin, hydroxypropylmethylcellulose (HPMC), or plastic.

In some embodiments, the capsule comprises medicament comprising one or more active pharmaceutical ingredients (APIs) alone or together with one or more pharmaceutically acceptable carriers. The active pharmaceutical ingredients and pharmaceutically acceptable carriers may be present in micronized, non-micronized form or mixtures thereof.

One or more active pharmaceutical ingredients (APIs) that can be used in the inventions selected from, analgesics, e.g., codeine, dihydromorphine, ergotamine, fentanyl or morphine; anginal preparations, e.g., diltiazem; antiallergics, e.g., cromoglycate (e.g. as the sodium salt), ketotifen or nedocromil (e.g. as the sodium salt); antiinfectives e.g., cephalosporins, penicillins, streptomycin, sulphonamides, tetracyclines and pentamidine; antihistamines, e.g., methapyrilene; anti-inflammatories, e.g., beclomethasone (e.g. as the dipropionate ester), fluticasone (e.g. as the propionate ester), flunisolide, budesonide, rofleponide, mometasone e.g. as the furoate ester), ciclesonide, triamcinolone (e.g. as the acetonide) or 6α,9α-difluoro-11β-hydroxy-16α-methyl-3-oxo-17α-propionyloxy-androsta-1,4-diene-17β-carbothioic acid S-(2-oxo-tetrahydro-furan-3-yl) ester; antitussives, e.g., noscapine; bronchodilators, e.g., albuterol (e.g. as free base or sulphate), salmeterol (e.g. as xinafoate), ephedrine, adrenaline, fenoterol (e.g. as hydrobromide), formoterol (e.g. as fumarate), isoprenaline, metaproterenol, phenylephrine, phenylpropanolamine, pirbuterol (e.g. as acetate), reproterol (e.g. as hydrochloride), rimiterol, terbutaline (e.g. as sulphate), isoetharine, tulobuterol or 4-hydroxy-7-[2-[[2-[[3-(2-phenylethoxy)propyl]sulfonyl]ethyl] amino]ethyl -2(3H)-benzothiazolone; adenosine 2a agonists, e.g. 2R,3R,4S,5R)-2-[6-Amino-2-(1S-hydroxymethyl-2-phenyl-ethylamino)-purin-9-yl]-5-(2-ethyl-2H-tetrazol-5-yl)-tetrahydro-furan-3,4-diol (e.g. as maleate);. α4 integrin inhibitors e.g. (2S)-3-[4-({[4-(aminocarbonyl)-1-piperidinyl]carbonyl}oxy)phenyl]-2-[((2S-)-4-methyl-2-{[2-(2 methylphenoxy)acetyl]amino}pentanoyl)amino]propanoic acid (e.g. as free acid or potassium salt), diuretics, e.g., amiloride; anticholinergics, e.g., ipratropium (e.g. as bromide), tiotropium (e.g. as bromide), atropine or oxitropium; hormones, e.g., cortisone, hydrocortisone or prednisolone; xanthines, e.g., aminophylline, choline theophyllinate, lysine theophyllinate or theophylline; therapeutic proteins and peptides, e.g., insulin or glucagon; vaccines, diagnostics, and gene therapies. It will be clear to a person skilled in the art that, where appropriate, the medicaments may be used in the form of salts, (e.g., as alkali metal or amine salts or as acid addition salts) or as esters (e.g., lower alkyl esters) or as solvates (e.g., hydrates) to optimise the activity and/or stability of the medicament.

In some embodiments, the medicament may in aspects, be a mono-therapy (i.e. single active medicament containing) product or it may be a combination therapy (i.e. plural active medicaments containing) product. Suitable medicaments or medicament components of a combination therapy product are typically selected from the group consisting of anti-inflammatory agents (for example a corticosteroid particularly inhaled corticosteroid (ICS) or an NSAID), anticholinergic agents (for example, an M1, M2, M1/M2 or M3 receptor antagonist particularly long-acting muscarinic antagonist (LAMA), other β2-adrenoreceptor agonists particularly long-acting β2-agonist (LABA), anti-infective agents (e.g. an antibiotic or an antiviral), and antihistamines. All suitable combinations are envisaged. The present invention also provides combination therapy product typically double or triple LAMA, LABA, ICS combinations.

Suitable anti-inflammatory agents include corticosteroids and NSAIDs. Suitable corticosteroids are those oral and inhaled corticosteroids and their pro-drugs which have anti-inflammatory activity. Examples include methyl prednisolone, prednisolone, dexamethasone, fluticasone propionate,6α,9α-difluoro-17α-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17α-propionyloxy-androsta-1,4-diene-17β-carbothioic acid S-(2-oxo-tetrahydro-furan-3S-yl) ester, beclomethasone esters (e.g. the 17-propionate ester or the 17,21-dipropionate ester), budesonide, flunisolide, mometasone esters (e.g. the furoate ester), triamcinolone acetonide, rofleponide, ciclesonide, butixocort propionate, RPR-106541, and ST-126. Preferred corticosteroids include fluticasone propionate, 6α,9α-difluoro-11β-hydroxy-16α-methyl-17α-[(-4-methyl-1,3-thiazole-5-carbonyl)oxy]-3-oxo-androsta-1,4-diene-17β-carbothioic acid S fluoromethyl ester and 6α,9α-difluoro-17α-[(2-furanylcarbonyl) oxy]-11β-hydroxy-16α-methyl-3-oxo-androsta-1,4-diene-17β-carbothioic acid S-fluoro methyl ester, more preferably 6α,9α-difluoro-17α-[(2-furanylcarbonyl)oxy]-11β-hydroxy-16α-methyl-3-oxo-androsta-1,4-diene-17β-carbothioic acid S-fluoromethyl ester.

Suitable NSAIDs include sodium cromoglycate, nedocromil sodium, phosphodiesterase (PDE) inhibitors (e.g. theophylline, PDE4 inhibitors or mixed PDE3/PDE4 inhibitors), leukotriene antagonists, inhibitors of leukotriene synthesis, iNOS inhibitors, tryptase and elastase inhibitors, β2integrin antagonists and adenosine receptor agonists or antagonists (e.g. adenosine 2a agonists), cytokine antagonists (e.g. chemokine antagonists) or inhibitors of cytokine synthesis. Suitable other β2-adrenoreceptor agonists include salmeterol (e.g. as the xinafoate), salbutamol (e.g. as the sulphate or the free base), formoterol (e.g. as the fumarate), fenoterol or terbutaline and salts thereof.

Suitable anticholinergic agents are those compounds that act as antagonists at the muscarinic receptor, in particular those compounds, which are antagonists of the M1 and M2 receptors. Exemplary compounds include the alkaloids of the belladonna plants as illustrated by the likes of atropine, scopolamine, homatropine, hyoscyamine; these compounds are normally administered as a salt, being tertiary amines.

Particularly suitable anticholinergics include ipratropium (e.g. as the bromide), sold under the name Atrovent, oxitropium (e.g. as the bromide) and tiotropium (e.g. as the bromide). Also of interest are: methantheline, propantheline bromide, anisotropine methyl bromide or Valpin 50, clidinium bromide, copyrrolate (Robinul), isopropamide iodide, mepenzolate bromide (U.S. Pat. No. 2,918,408), tridihexethyl chloride, and hexocycliummethylsulfate. See also cyclopentolate hydrochloride, tropicamide, trihexy-phenidyl hydrochloride, pirenzepine, telenzepine, or methoctramine, and the compounds disclosed in WO01/04118.

Suitable antihistamines (also referred to as H1-receptor antagonists) include any one or more of the numerous antagonists known which inhibit H1-receptors, and are safe for human use. All are reversible, competitive inhibitors of the interaction of histamine with H1-receptors. Examples include ethanolamines, ethylenediamines, and alkylamines. In addition, other first generation antihistamines include those which can be characterized as based on piperizine and phenothiazines. Second generation antagonists, which are non-sedating, have a similar structure-activity relationship in that they retain the core ethylene group (the alkylamines) or mimic the tertiary amine group with piperizine or piperidine. Exemplary antagonists are as follows: Ethanolamines: carbinoxamine maleate, clemastine fumarate, diphenylhydramine hydrochloride, and dimenhydrinate. Ethylenediamines: pyrilamineamleate, tripelennamine HCl, and tripelennamine citrate. Alkylamines: chlropheniramine and its salts such as the maleate salt, and acrivastine. Piperazines: hydroxyzine HCl, hydroxyzine pamoate, cyclizine HCl, cyclizine lactate, meclizine HCl, and cetirizine HCl. Piperidines: Astemizole, levocabastine HCl, loratadine or its descarboethoxy analogue, and terfenadine and fexofenadine hydrochloride or another pharmaceutically acceptable salt.

Azelastine hydrochloride is yet another H1receptor antagonist which may be used in combination with a PDE4 inhibitor. Particularly suitable anti-histamines include methapyrilene and loratadine. In respect of combination products, co-formulation compatibility is generally determined on an experimental basis by known methods and may depend on chosen type of medicament dispenser action.

The medicament components of a combination product are suitably selected from the group consisting of anti-inflammatory agents (for example a corticosteroid or an NSAID), anticholinergic agents (for example, an M1, M2, M1/M2 or M3 receptor antagonist), other β2-adrenoreceptor agonists, anti-infective agents (e.g. an antibiotic or an antiviral), and antihistamines. All suitable combinations are envisaged.

Typically, the co-formulation compatible components comprise a β2-adrenoreceptor agonist and a corticosteroid; and the co-formulation incompatible component comprises a PDE-4 inhibitor, an anti-cholinergic or a mixture thereof. The β2-adrenoreceptor agonists may for example be salbutamol (e.g., as the free base or the sulphate salt) or salmeterol (e.g., as the xinafoate salt) or formoterol (e.g. as the fumarate salt). The corticosteroid may for example, be a beclomethasone ester (e.g., the dipropionate) or a fluticasone ester (e.g., the propionate) or budesonide.

In one example, the co-formulation compatible components comprise fluticasone propionate and salmeterol, or a salt thereof (particularly the xinafoate salt) and the co-formulation incompatible component comprises a PDE-4 inhibitor, an anti-cholinergic (e.g. ipratropium bromide or tiotropium bromide) or a mixture thereof.

In another example, the co-formulation compatible components comprise budesonide and formoterol (e.g. as the fumarate salt) and the co-formulation incompatible component comprises a PDE-4 inhibitor, an anti-cholinergic (e.g. ipratropium bromide or tiotropium bromide) or a mixture thereof.

It is intended that the scope of the present invention herein disclosed should not be limited by any particular embodiment described herein. While various embodiments of the present invention have been described above, it should be noted that they have been presented by way of example only, and not limitation. Numerous changes to the disclosed embodiments can be made in accordance with the disclosure herein without departing from the spirit or scope of the invention.

### EXAMPLES

The following examples are included to demonstrate particular embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

1. Weighed quantity of Tiotropium bromide along with lactose monohydrate are sifted through 60# SS Sieve.

2. The blend of step 1 is loaded in in High Shear Blender (Pharmaconnect TRV) and mixed for 3 minutes.

3. The blend of step 2 is filed into empty HPMC capsules using suitable capsule filling machine.

The capsule rotation profiles were generated using Next Generation Impactor (NGI) after two inhalations each from 5 capsules containing Tiotropium bromide, each at 39 L/min for 6.2 seconds (equivalent to 4 L air volume). Table 1 and 2 demonstrate that continuous rotation of capsules containing Tiotropium bromide is obtained by using mesh of Polyoxymethylene. Analysis result under given conditions are as follows:

TABLE 1
Capsule rotation behavior of Inhaler with different Mesh materials

| | Capsule chamber material | | Capsule # | | | | |
|---|---|---|---|---|---|---|---|
| Mesh material | (Material of Construction) | Inhalation # | Cap 1 | Cap 2 | Cap 3 | Cap 4 | Cap 5 |
| Acrylonitrile Butadiene | Acrylonitrile | First | CR¹ | NCR² | NCR | NCR | NCR |
| Styrene (ABS) | Butadiene Styrene (ABS) | Second | NCR | NCR | NCR | NCR | NCR |
| Acrylonitrile Butadiene | | First | NCR | NCR | NCR | NCR | NCR |
| Styrene (ABS) + | | Second | NCR | NCR | NCR | NCR | NCR |
| Antistatic agent 1.5% | | | | | | | |
| Polypropylene (PP) | | First | CR | NCR | NCR | NCR | NCR |
| | | Second | NCR | NCR | NCR | NCR | NCR |
| Methacrylate | | First | NCR | NCR | NCR | NCR | NCR |
| Butadiene Styrene (MBS) | | Second | NCR | NCR | NCR | NCR | NCR |
| Polyoxy Methylene (POM) | | First | CR | CR | CR | CR | CR |
| | | Second | CR | CR | CR | CR | CR |

¹CR: Continuous Rotation,
²NCR: Non continuous rotation

TABLE 2
Capsule rotation behavior of Inhaler of polyoxymethylene(POM)
mesh with different capsule chamber materials

| Capsule Chamber material | Mesh | | Capsule # | | | | |
|---|---|---|---|---|---|---|---|
| (Material of Construction) | (Material of Construction) | Inhalation # | Cap 1 | Cap 2 | Cap 3 | Cap 4 | Cap 5 |
| Acrylonitrile Butadiene | Polyoxy methylene (POM) | First | CR¹ | CR | CR | CR | CR |
| Styrene (ABS) | | Second | CR | CR | CR | CR | CR |
| Acrylonitrile Butadiene | | First | CR | CR | CR | CR | CR |
| Styrene (ABS) + | | Second | CR | CR | CR | CR | CR |
| Antistatic agent 1.5% | | | | | | | |
| Copolyster | | First | CR | CR | CR | CR | CR |
| | | Second | CR | CR | CR | CR | CR |
| Polypropylene | | First | CR | CR | CR | CR | CR |
| | | Second | CR | CR | CR | CR | CR |
| Methacrylate Butadiene | | First | CR | CR | CR | CR | CR |
| Styrene (MBS) | | Second | CR | CR | CR | CR | CR |
| Polyethylene | | First | CR | CR | CR | CR | CR |
| Terephthalate (PET) | | Second | CR | CR | CR | CR | CR |

¹CR: Continuous Rotation

TABLE 3
Deposition profile of Tiotropium Bromide from Inhaler with two
different mesh material using Next Generation Impactor (NGI)
The Aerodynamic Particle Size Distribution (APSD) profiles
were generated using Next Generation Impactor (NGI) device,
after two inhalations each from 5 capsules containing
Tiotropium bromide, each at 39 L/min for 6.2 seconds
(equivalent to 4 L air volume). Deposition on Individual
stages of the NGI measured by extracting the deposited
Tiotropium bromide separately in suitable diluent and
quantified using HPLC method. Impactor sized mass (ISM),
Fine particle dose and fine particle fraction calculated
using CITDAS software. Analysis results under given
conditions are as follows:
% LC: % Label claim 10.4 ug

| | Deposition profile | |
|---|---|---|
| | | Inhaler with |
| | | mesh material |
| | Inhaler with mesh material | of Polyoxy |
| | of Acrylonitrile Butadiene | Methylene |
| | Styrene (ABS) | (POM) |
| | Mean Deposition (n = 3) | |
| Mouthpiece & Throat | 3.09 | 4.07 |
| (MP/T) - μg | | |
| Pre Separator(PS) - μg | 2.52 | 2.35 |
| Stage 1 - μg | 0.32 | 0.37 |
| Stage 2 - μg | 0.68 | 1.00 |
| Stage 3 - μg | 1.32 | 1.96 |
| Stage 4 - μg | 1.51 | 1.88 |
| Stage 5 - μg | 0.59 | 0.57 |
| Stage 6 - μg | 0.15 | 0.16 |
| Stage 7 - μg | 0.05 | 0.07 |
| Micro orifice collector | 0.00 | 0.00 |
| (MOC) - μg | | |
| Impactor Sized Mass | 4.30 | 5.64 |
| (ISM) - μg | | |
| Impactor Sized Mass (% | 41.3 | 54.2 |
| LC) | | |
| Fine Particle Dose (FPD) - | 3.34 | 4.24 |
| μg | | |
| Fine Particle Fraction (% | 32.1 | 40.8 |
| LC) | | |

The inhaler with mesh material of Polyoxymethylene results in increase of Fine Particle Dose and Fine Particle Fraction in deposition profile.

1. An inhaler device for dispensing medicament comprising: a dispenser flow channel leading to a mouthpiece, a medicament holder, and a mesh part placed in the dispenser flow channel between the medicament holder and the mouthpiece, wherein said mesh part is made of a Polyoxymethylene.

2. The inhaler device according to claim 1, wherein the medicament comprises one or more active pharmaceutical ingredient(s) alone or together with one or more excipients or pharmaceutically acceptable carrier(s).

3. The inhaler device according to claim 2, wherein the active pharmaceutical ingredient is selected from group consisting of anticholinergics, anti-inflammatories, analgesics, anti-anginal, anti-allergic, antihistamines, antitussives, bronchodilators, anti-infectives, leukotriene inhibitors, PDE IV inhibitors, antitussives, diuretics, hormones, cromolyns, therapeutic proteins and peptides, vaccines, diagnostics and gene therapies or combinations thereof.

4. The inhaler device according to claim 3, wherein said anticholinergic is tiotropium bromide.

5. The inhaler device according to claim 1, wherein the medicament holder is designed to hold a capsule containing dry powder medicament.

6. The inhaler device according to claim 4, wherein the dry powder medicament is used for the treatment of asthma, chronic obstructive pulmonary disease (COPD), bronchitis or chest infection.
